(19) United States
(12) Patent Application Publication
(10) Pub. No.: US 2003/0069232 A1
(43) Pub. Date: Apr. 10, 2003

(54) Topical treatment of ocular hypertension, glaucoma, ischemic retinopathy and age-related macular degeneration with ophthalmic formulation of dopamine antagonists

(76) Inventor: George C.Y. Chiou, College Station, TX (US)

Correspondence Address: MORRISON & FOERSTER LLP, 755 PAGE MILL RD, PALO ALTO, CA, 94304-1018, US

(21) Appl. No.: 09/796,987

(22) Filed: Feb. 28, 2001

Related U.S. Application Data
(63) Continuation of application No. 09/425,628, filed on Oct. 22, 1999, now abandoned.

(51) Int. Cl. A61K 31/551; A61K 31/541; A61K 31/137

(52) U.S. Cl. 514/224.8; 514/649; 514/220

(57) ABSTRACT

This invention provides ocular formulations comprising an ocular drug and a carboxylic acid in an amount sufficient to maintain the pH of the formulation from about 4.5 to about 7.5. The ocular drug may be a dopamine antagonist and the acid may be lactic acid, citric acid or tartaric acid. In some aspects, the pH of the formulation is about 5.5 The ocular formulations of this invention provide enhanced bioavailability which results in increased drug concentrations across the cornea and in the eye ball, i.e., aqueous humor and intraocular organs and chambers. Moreover, the present formulations are non-irritating when applied topically and have a shelf-life of at least fourteen days at 25° C. Methods are also provided to increase ocular blood flow by using present ocular formulations comprising dopamine antagonists or other drugs for the prevention and treatment of ocular hypertension, glaucoma, ischemic retinopathy and age-related macular degeneration (AMD).

### TECHNICAL FIELD

The present invention relates generally to ocular formulations and methods for using those formulations to improve blood flow to the retina and choroid to halt or reverse the course of visual deterioration. Accordingly, this invention transcends the related disciplines of pharmaceutical sciences, ocular pharmacology and medicine.

### BACKGROUND OF THE INVENTION

Several potential drugs have been developed with high anticipation of treating various eye diseases, yet only a few of those potential drugs have reached the clinics because of the problems of drug delivery. Ocular drug delivery faces three major difficulties: first, the ocular bioavailability of the drug is often poor because the drug needs to cross the cornea to enter the eye ball, i.e., the aqueous humor and other interior anatomical organs of the eye; second, very often, the drug formulation is irritable when applied topically to the eye; and third, the ocular formulations are very unstable, i.e., have a short shelf-life, in the order of a few days to few weeks. For example, most, if not all dopamine antagonists do not dissolve in plain aqueous medium and, as a result, their non-aqueous formulations often produce severe eye irritation. Various absorption enhancers and anti-irritants have been proposed in the prior art to overcome these difficulties. However, the search for a successful resolution to the problem continues.

Accordingly, there is a need for stable ocular formulations that enhance the ocular bioavailability of a drug with reduced ocular irritation when administered topically. As the following description illustrates, the present invention meets this need.

### DISCLOSURE OF THE INVENTION

A formulation for ocular delivery is provided wherein the formulation comprises an ocular drug and a carboxylic acid in an amount sufficient to maintain the pH of the formulation from about 4.5 to about 7.5. The ocular drug may be a dopamine antagonist. Additionally, the formulation may also comprise an adjuvant. The carboxylic acid can be a hydroxymonocarboxylic acid having the following chemical formula:

R₁(CR₂OH)ₘ(CH₂)ₙCOOH,

or a hydroxydicarboxylic acid having the following formula:

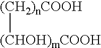

or, a hydroxyacid having the following formula:

R(OH)ₘ(COOH)ₙ

R, R₁ and R₂ are selected from the group consisting of hydrogen, alkyl, aralkyl and aryl group, wherein

the alkyl, aralkyl and aryl groups may be saturated or unsaturated, and straight or branched, and the alkyl group has from 1 to 25 carbon atoms, the aralkyl group has from 7 to 25 carbon atoms, and the aryl group has from 6 to 25 carbon atoms;

m is an integer of from 1 to 9, and n is an integer of from 0 to 23 when the acid is a monohydroxycarboxylic acid and from 1 to 9 when the acid is a hydroxyacid, or a D, L and DL isomer, or a mixture thereof.

One specific example is a formulation for ocular delivery comprising a dopamine antagonist, a carboxylic acid in an amount sufficient to maintain the pH of the formulation from about 4.5 to about 7.5, wherein the dopamine antagonist is metoclopromide, loxapine, or droperidol, and the acid is tartaric acid, lactic acid or citric acid and the pH of the formulation is about 5.5.

The formulation may be in a solution, dispersion, cream, ointment, gel, or film. The formulation has a shelf-life of at least 14 days at 25° C.

A method is also provided to increase blood flow to the retina or choroid, to reduce intraocular pressure, or to treat or prevent visual deterioration associated with decreased retinal or choroidal blood flow or increased intraocular pressure. The method comprises ocularly administering a formulation comprising a therapeutically effective amount of a dopamine antagonist, a carboxylic acid as described above in an amount sufficient to maintain the pH of the formulation from about 4.5 to about 7.5 to a subject having decreased retinal or choroidal blood flow or increased intraocular pressure.

The decreased retinal or choroidal blood flow may be due to low pressure glaucoma, ischemic retinal degeneration, or age-related macular degeneration.

### BRIEF DESCRIPTION OF DRAWINGS

FIG. 1 is a graphical display of stability data of droperidol formulation comprising citric acid.

FIG. 2 is a graphical display of stability data of droperidol formulation comprising tartaric acid.

FIG. 3 is a graphical display of stability data of droperidol formulation comprising citric acid as determined for 16 days.

FIG. 4 is a graphical display of stability data of droperidol formulation comprising tartaric acid as determined for 16 days.

### MODES FOR CARRYING OUT THE INVENTION

A. General Techniques

One of ordinary skill in the art would readily appreciate that the formulations and methods described herein can be prepared and practiced by using known procedures in the pharmaceutical arts. Thus, the practice of the present invention employs, unless otherwise indicated, conventional techniques of pharmaceutical sciences including pharmaceutical dosage form design, drug development, pharmacology, of organic chemistry, and polymer sciences. See generally, for example, Remington: The Science and Practice of Pharmacy, 19th Ed., Mack Publishing Co., Easton, Pa. (1995) (hereinafter REMINGTON).

B. Definitions

As used herein, certain terms have the following defined meanings.

As used in the description and claims, the singular forms a, an and the include plural references unless the context clearly dictates otherwise. For example, the term a drug may refer to one or more drugs for use in the presently disclosed invention.

The term ocular refers to the eye, including all its muscles, nerves, blood vessels, tear ducts, membranes etc., as well as structures that are immediately connected with the eye, and its physiological functions. The terms ocular, ocular structures and eye are used interchangeably throughout this disclosure.

The term ocular bioavailability as used herein refers to the extent of the dosage that is topically applied to the eye that is available to the ocular tissues, organs and structures that are posterior or interior to the cornea. The drug reaches these tissues, organs and structures by passing through the cornea.

Ocular delivery refers to the delivery of a desired drug to the eye. In some aspects, ocular delivery may include systemic delivery through the eye, because, as one of ordinary skill in the art recognizes, a localized delivery to a particular site in the eye may result, due to the highly perfused nature of the eye, in the drug being absorbed through the blood vessels and carried to a location remote from the eye leading to systemic delivery. Given this characteristic, it may be advantageous in some cases to aim for systemic delivery through the eye. Such systemic delivery is also within the scope of the present invention.

The term drug device or delivery device or simply device as used herein refers to a composition that contains and or delivers a drug to a subject and the composition is generally considered to be otherwise pharmacologically inactive.

The term drug includes any known pharmacologically active agent as well as its pharmaceutically acceptable salt, prodrug such as an ester or an ether, or a salt of a prodrug, or a solvate such as ethanolate, or other derivative of such pharmacologically active drug. These salts, prodrugs, salts of prodrugs, solvates and derivatives are well-known in the art.

Salts of the pharmacologically active drugs may be derived from inorganic or organic acids and bases. Examples of inorganic acids include hydrochloric, hydrobromic, sulfuric, nitric, perchloric, and phosphoric acids. Examples of bases include alkali metal (e.g., sodium) hydroxides, alkaline earth metal (e.g., magnesium) hydroxides, ammonia, and compounds of formula NW₄⁺, wherein W is C₁₋₄ alkyl.

Examples of organic salts include: acetate, propionate, butyrate, hexanoate, heptanoate, undecanoate, palmoate, cyclopentanepropionate, adipate, alginate, aspartate, benzoate, citrate, oxalate, succinate, tartarate, lactate, maleate, fumarate, camphorate, nicotinate, pectinate, picrate, pivalate, tosylate, gluconate, digluconate, hemisulfate, methanesulfonate, ethanesulfonate, 2-hydroxyethanesulfonate, dodecylsulfate, camphorsulfonate, benzenesulfonate, 2-naphthalenesulfonate, thiocyanate, phosphate, glycerophosphate, and phenylpropionate. Several of the officially approved salts are listed in REMINGTON, supra, Chapter 83.

The term derivative of a compound as used herein means a chemically modified compound wherein the chemical modification takes place at one or more functional groups of the compound and /or on an aromatic, alicyclic, or heterocyclic structures, when present. The derivative however is expected to retain the pharmacological activity of the compound from which it is derived.

The term prodrug refers to a precursor of a pharmacologically active compound wherein the precursor itself may or may not be pharmacologically active but, upon administration, will be converted, either metabolically or otherwise, into the pharmacologically active drug of interest. Several prodrugs have been prepared and disclosed for a variety of pharmaceuticals. See, for example, Bundgaard, H. and Moss, J., J. Pharm. Sci. 78: 122-126 (1989). Thus, one of ordinary skill in the art knows how to prepare these derivatives and prodrugs with commonly employed techniques of organic synthesis.

In addition, polymorphs, isomers (including stereoisomers, geometric isomer and optical isomers) and anomers of the drugs described herein are contemplated.

The terms drug and pharmaceutical as used herein are identical in meaning and thus are used interchangeably.

An adjuvant is an agent that may affect any of (1) the rate of release of the drug; (2) the stability of the drug; (3) the solubility of the drug; or (4) physicochemical characteristics of the formulation, including pH, osmotic pressure, etc. Thus, adjuvants may include solubilizing agents, solubility decreasing agents, dispersing agents, preservatives, viscosity enhancers, absorption enhancers, and stabilizing agents.

A solubilization agent increases the solubility of a pharmaceutical in the formulation. The solubilization agent preferably comprises between about 0.01% and about 20% by weight of the final formulation, and more preferably between about 0.1% and 10% by weight of the final formulation.

A solubility decreasing agent can be used in the formulation to achieve the desired release characteristics. Solubility of a drug can be decreased by techniques known in the art, such as by complexation, etc. Examples of complexation agents include: 2-hydroxynicotinic acid, 2-hydroxyphenylacetic acid, cyclodextrans, phthalic acid, polyethylene glycols, hydroquinone and derivatives thereof, caffeine, bile salts and acids.

As used herein, the term solubility refers to the extent to which a solute dissolves in a solvent, wherein the solute and “solvent” may be of the same or of different physical state. Thus, a solution of a solid or a liquid in any “solvent” such as a solid, liquid or gas is within the scope of this term.

Solubility can be expressed in many ways, such as: weight/volume (grams/mL); molality (number of moles of solute/1000 grams of solvent); mol fraction (fraction of the total number of mols present which are mole of one component); mol % (mol fraction×100); normality (number of gram equivalent weights of solute dissolved in 1000 mL of solution); % by weight (% w/w); % weight in volume (%w/v); % by volume (% v/v).

Solubility can also be described by terms such as: very soluble (less than 1 part of solvent per 1 part of solute); freely soluble (from 1 to 10 parts of solvent per 1 part of solute); soluble (from 10 to 30 parts of solvent per 1 part of solute); sparingly soluble (from 30 to 100 parts of solvent for 1 part of solute); slightly soluble (from 100 to 1000 parts of solvent for 1 part of solute); very slightly soluble (from 1000 to 10,000 parts of solvent for 1 part of solute); and practically insoluble, or insoluble (more than 10,000 parts of solvent for 1 part of solute). For further elaboration, see REMINGTON, supra, Chapter 16, which is incorporated by reference.

A dispersing agent is an agent that facilitates the formation of a dispersion of one or more internal phases in a continuous phase. Examples of such dispersions include suspensions and emulsions, wherein the continuous phase may be water, for example, and the internal phase is a solid or a water-immiscible liquid, respectively. Thus, dispersing agents may include suspending agents and emulsifying agents.

An effective amount is an amount sufficient to effect beneficial or desired therapeutic results such as prevention or treatment of visual deterioration. An effective amount can be administered in one or more administrations, applications or dosages. Determination of an effective amount for a given administration is well within the ordinary skill in the pharmaceutical arts.

Administration refers to a method of ocularly placing a formulation such that the drug provided in the formulation brings out the desired therapeutic effect. The placing of the formulation can be by any pharmaceutically accepted means such as instilling, applying, rubbing, dropping, spraying, rolling, squeezing, spreading, etc. These and other methods of administration are known in the art.

The term pharmaceutically acceptable is an adjective and means that the ingredient that is being qualified is compatible with the other ingredients of the formulation and not injurious to the patient. Several pharmaceutically acceptable ingredients are known in the art and official publications such as THE UNITED STATES PHARMACOEPIA describe the analytical criteria to assess the pharmaceutical acceptability of numerous ingredients of interest.

Decreased bloodflow as used herein refers to choroidal or retinal blood flow that is below normal human retinal blood flow. Normal blood flow has been reported in the range of 8.1 to 18.5 μl/min.

Treatment as used herein refers to the reduction or elimination of visual deterioration resulting from decreased blood flow to the retina and choroid (therapy).

Prevention refers to the treatment of patients with decreased retinal and/or choroidal blood flow to avoid visual deterioration (prophylaxis).

Formulation refers to a composition comprising a drug. Formulation also comprises a pharmaceutically acceptable carrier which is generally considered to be pharmacologically inactive.

Therapeutically effective amount refers to an amount of a pharmaceutically active substance useful in the prevention or treatment of visual deterioration.

The term shell-life as used herein refers to the time needed for a drug concentration in a formulation to decrease to 90% of the initial concentration. This shelf-life is designated as t₉₀, and is stated at 25° C. Accelerated stability tests can be performed at higher temperatures and the data can be approximated for 25° C. Methods to perform these tests are well-known in the art. See Remington, supra, Chapter 18.

Ischemic retinal degeneration is the degeneration of the retina and occurs as a result of the impairment or interruption of the supply of oxygen or other nutrients to the retina via the central retinal artery or to the choroid via the posterior ciliary artery. Such impairment or interruption may result from various diseases and conditions such as diabetic retinopathy, glaucoma, sickle cell retinopathy, vascular abnormalities, obstructive arterial and venous retinopathies, venous capillary insufficiency, hypertensive retinopathy, inflammation, tumors, and retinal detachment.

The term dopamine antagonist refers to a drug that is an antagonist to all subtypes of central and peripheral dopaminergic receptors. The dopamine antagonists include, but not limited to, phenothiazines, thioxanthenes, butyrophenones, dihydroindolones, dibenzoxazepines, dibenzodiazepines. Representative examples of these dopamine antagonists include: acetophenazine, chlorpromazine, clozapine, chlorprothixene, droperidol, ergoloid, fluphenazine, haloperidol, loxapine, mesoridazine, molindone, perphenazine, pimozide, promazine, thioridazine, thiothixene, trifluoperazine, and metolcopramide.

Concentrations, amounts, pH values, etc., of various aspects of this invention are often presented in a range format throughout this application. The description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as a pH of 4.5 to 7.5 should be considered to have specifically disclosed subranges such as 4.5 to 6.0, 4.5 to 7.0, 5.0 to 7.0, 5.0 to 7.5, 5.5 to 7.5, etc., as well as individual numbers within that range, such as, 4.6, 4.8, 5.3, 5.6, 5.9, 6.2, 6.6, 7.2, 7.4 etc. This construction applies regardless of the breadth of the range and in all contexts throughout this disclosure.

C. The Invention

The present invention is based on the finding that ocular bioavailability of a drug can be significantly enhanced by formulations with an optimal pH range and comprising certain adjuvants to help the drug cross the cornea and thus provide a greater concentration of the drug in the eye ball, wherein the formulation does not cause substantial eye irritation. Thus, these formulations provide a convenient means for treating or preventing ocular conditions and disorders.

Methods are also provided to increase blood flow to the retina or choroid in a subject with decreased retinal and choroidal blood flow comprising administering a formulation of the present invention.

In one aspect, the formulations of the present invention increase blood flow to the retina for the treatment of low pressure glaucoma, for the prevention of ischemic retinal degeneration, or to prevent or treat visual deterioration associated with decreased choroidal or retinal blood flow.

In a further embodiment, the invention relates to an ocular delivery device comprising a dopamine antagonist.

D. The Formulation

The ocular formulation of the present invention comprises an ocular drug and a carboxylic acid in an amount sufficient to maintain the pH of the formulation from about 4.5 to about 7.5.

1. The Ocular Drugs

Practically any ocular drug whose formulation causes irritation upon topical administration to the eye and whose formulation has poor ocular bioavailability can be used in the present formulation for its ocular delivery. Several such drugs are well-known in the art. For example, most dopamine antagonists are water insoluble and their ocular formulations cause severe irritation when applied topically to the eye. Moreover, the formulations have poor ocular bioavailability.

Dopamine antagonists comprise a diverse category of chemical classes known as, for example, phenothiazines, thioxanthenes, butyrophenones, dihydroindolones, dibenzoxazepines, and dibenzodiazepines. Representative examples of these dopamine antagonists include: acetophenazine, chlorpromazine, clozapine, chlorprothixene, droperidol, ergoloid, fluphenazine, haloperidol, loxapine, mesoridazine, molindone, perphenazine, pimozide, promazine, thioridazine, thiothixene, trifluoperazine, and metolcopramide butyrophenone or a phenothiazine or a mixture thereof.

In one aspect, the dopamine antagonist is droperidol, loxapine, or a mixture thereof. In another aspect, the drug for use in the present invention is metoclopromide.

Additional categories of ocular drugs that can be delivered using the formulations of the present invention include: anesthetics, analgesics, cell transport/mobility impending agents such as colchicine, vincristine, cytochalasin B and related compounds; antiglaucoma drugs including beta-blockers such as timolol, betaxolol, atenolol, etc; carbonic anhydrase inhibitors such as acetazolamide, methazolamide, dichlorphenamide, diamox; and neuroprotectants such as nimodipine and related compounds.

Additional examples include antibiotics such as tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole and sulfisoxazole; anti-fungal agents such as fluconazole, nitrofurazone, ketoconazole, and related compounds; anti-viral agents such as trifluorothymidine, acyclovir, ganciclovir, DDI, AZT, foscarnet, vidarabine, trifluorouridine, idoxuridine, ribavirin, protease inhibitors and anti-cytomegalovirus agents; antiallergenics such as methapyriline, chlorpheniramine, pyrilamine and prophenpyridamine; anti-inflammatories such as hydrocortisone, dexamethasone, fluocinolone, prednisone, prednisolone, methylprednisolone, fluoromethalone, betamethasone and triaminolone; decongestants such as phenylephrine, naphazoline, and tetrahydrazoline; miotics and anti-cholinesterases such as pilocarpine, carbachol, di-isopropyl fluorophosphate, phospholine iodine, and demecarium bromide; mydriatics such as atropine sulfate, cyclopentolate, homatropine, scopolamine, tropicamide, eucatropine; sympathomimetics such as epinephrine and vasoconstrictors and vasodilators. Anticlotting agents such as heparin, antifibrinogen, fibrinolysin, anticlotting activase, etc., can also be delivered.

Antidiabetic agents that may be delivered using the present formulations include acetohexamide, chlorpropamide, glipizide, glyburide, tolazamide, tolbutamide, insulin, aldose reductase inhibitors, etc. Some examples of anti-cancer agents include 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamistole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil mustard, vinblastine, vincristine and vindesine.

2. The Acids

In one aspect, the formulations of this invention comprise a carboxylic acid, which can be a hydroxymonocarboxylic acid having the following structure:

R₁(CR₂ OH)ₘ(CH₂)ₙCOOH, wherein

wherein m is an integer of from 1 to 9, and n is an integer of from 0 to 23, and each of R₁ and R₂ is independently hydrogen, alkyl, aralkyl and aryl, wherein

the alkyl, aralkyl and aryl groups may be saturated or unsaturated, and straight or branched and the alkyl group has from 1 to 25 carbon atoms, the aralkyl group has from 7 to 25 carbon atoms, and the aryl group has from 6 to 25 carbon atoms;

The typical alkyl, aralkyl and aryl groups for R₁ and R₂ include methyl, ethyl, propyl, isopropyl, benzyl and phenyl. The acids of the invention include D, L and DL isomers of one of the above acids or a mixture thereof.

More specifically, the hydroxymonocarboxylic acid may be a: glycolic acid, lactic acid, methyllactic acid, 2-hydroxybutanoic acid, mandelic acid, atrolactic acid, phenyllactic acid, glyceric acid, 2,3,4-trihydroxybutanoic acid, 2,3,4,5-tetrahydroxypentanoic acid, 2,3,4,5,6-pentahydroxyhexanoic acid, 2-hydroxydodecanoic acid, 2,3,4,5,6,7-hexahydroxyheptanoic acid, benzillic acid, 4-hydroxymandelic acid, 4-chloromandelic acid, 3-hydroxybutanoic acid, 4-hydroxybutanoic acid, 2-hydroxyhexanoic acid, 5-hydroxydodecanoic acid, 12-hydroxydodecanoic acid, 10-hydroxydecanoic acid 16-hydroxyhexadecanoic acid, 2-hydroxy-3-methylbutanoic acid, 2-hydroxy-4-methylpentanoic acid, 3-hydroxy-4-methoxymandelic acid, 4-hydroxy-3-methoxymandelic acid, 2-hydroxy-2-methylbutanoic acid, 3-(2-hydroxyphenyl) lactic acid, 3-(4-hydroxyphenyl) lactic acid, hexahydromandelic acid, 3-hydroxy-3-methylpentanoic acid, 4-hydroxydecanoic acid, 5-hdroxydecanoic acid and aleuritic acid, or a D, L and DL isomers of one of the above acids or a mixture thereof.

In another aspect, the formulations of this invention comprise a hydroxydicarboxylic acid having the following formula:

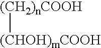

wherein m is an integer of from 1 to 9, and n is an integer of from 0 to 23, or a D, L and DL isomer or a mixture thereof.

In some aspects, the hydroxydicarboxylic acid can be a: tartronic acid, malic acid, tartaric acid, arabiraric acid, ribaric acid, xylaric acid, lyxaric acid, saccharic acid, mucic acid, mannaric acid, gularic acid, allaric acid, altraric acid, idaric acid and talaric acid, or a D, L and DL isomers of one of the above acids or a mixture thereof.

In some aspects, the formulations comprise a hydroxyacid having the following formula:

R(OH)ₘ(COOH)ₙ

wherein m is an integer of from 1 to 9, n is an integer of from 1 to 9, and R is a hydrogen, alky, aralkyl or aryl, wherein

the alkyl, aralkyl and aryl groups may be saturated or unsaturated, and straight or branched and the alkyl group has from 1 to 25 carbon atoms, the aralkyl group has from 7 to 25 carbon atoms, and the aryl group has from 6 to 25 carbon atoms;

The typical alkyl, aralkyl and aryl groups for R₁ and R₂ include methyl, ethyl, propyl, isopropyl, benzyl and phenyl. The acid includes a D, L and DL isomer of the above acids, or a mixture thereof.

Specific examples of the hydroxyacid include: citric acid, isocitric acid, citramalic acid, agaricic acid, quicnic acid, glucuronic acid, galacturonic acid, hydroxypyruvic acid, ascorbic acid, dihydroascorbic acid, dihydroxytartaric acid, 2-hydroxy-2-methylbutanoic acid, 1-hydroxy-1-cyclopropane carboxylic acid, 3-hydroxy-2-aminopentanoic acid, tropic acid, 4-hydroxy-2,2-diphenylbutanoic acid, 3-hydroxy-3-methylglutaric acid and 4-hydroxy-3-pentenoic acid, or a D, L and DL isomers of one of the above acids or a mixture thereof.

The desired characteristics of optimal pH, enhanced ocular bioavailability, and reduced irritation can be achieved by using any acids described above or a mixture thereof.

The pH of a formulation affects the overall formulation in at least two ways: a) by influencing the drugs' bioavailability by altering the ratio of the ionized versus nonionized amounts of the drug, and b) by potentially contributing to ocular irritation. For a drug to be ocularly bioavailable, the drug must penetrate the cornea to enter the eyeball. Therefore, ideally, the drug has to be both lipophilic and hydrophilic, i.e., the drug should be in a nonionized form in order to penetrate across the corneal epithelium, but it should also be water-soluble in order to move across the thick stroma. With weak acidic or alkalinic drugs, changes of pH can alter the ratio of nonionized vs. ionized molecules markedly and, thus, alter the ability to penetrate across the cornea.

The biochemical composition of cornea is not analogous to some other physiological barriers, such as skin. For example, the skin has a somewhat hydrophilic layer followed by a lipid bilayer, whereas the cornea comprises a lipid layer followed by hydrophilic stroma. Thus, the traditional teachings from dermal arts as to pH effects on permeation and bioavailability do not provide sufficient guidance for ocular drug delivery development.

One prevailing general practice in the art of ocular drug delivery has been to adjust the pH of the ocular formulation to a value close to 7.4, which is the normal ocular pH. Buffers or ion-exchange membrane devices have been used to adjust the pH of the formulation from about 7.0 to 7.4. However, the present inventor has discovered that formulations having a much lower pH can also be well-tolerated by the eye. Moreover, it has been discovered that the particular combination of a drug with an acid as described above at a pH of about 5.5 has resulted in significantly higher ocular bioavailability.

The acid can be present in sufficient amount to achieve a desired pH within the range of from about 4.5 to about 7.5. In some aspects, the amount of acid is in sufficient concentration such that the formulation achieves a desired pH from about 5.0 to about 6.0. In another aspect, the pH of the formulation is from about 5.2 to about 5.7. In a further aspect, the pH of the formulation is about 5.5. The exact amount or concentration of the acid required depends on the acid or mixture of acids selected and on the pH desired. However, such determination is within the ordinary skill in the art.

Thus, in one aspect, a formulation for ocular delivery comprises a dopamine antagonist, a carboxylic acid in an amount sufficient to maintain the pH of the formulation from about 4.5 to about 7.5, wherein the dopamine antagonist is metoclopromide, loxapine, or droperidol, and the acid is tartaric acid, lactic acid or citric acid and the pH of the formulation is about 5.5.

3. Adjuvants

Additionally, the formulations of this invention may comprise adjuvants that are known in the pharmaceutical arts. Some examples of adjuvants include: a viscosity enhancer, a preservative, a tonicity adjuster, an absorption enhancer, a stabilizer, or a mixture thereof.

The tonicity is important because hypotonic eye drops cause an edema of the cornea, and hypertonic eye drops cause deformation of the cornea. The ideal tonicity is approximately 300 mOsM. The tonicity of the present formulations can be achieved by methods described in Remington, supra, Chapter 17.

All ophthalmic solutions, except unit-dose preparations, are packaged in multiple-dose containers used for repeated instillations of eye drops into the eyes. Although the containers are sterilized, sealed, and tamper-proofed, they are easily contaminated once the lid is opened. Therefore, preservative are used to suppress the growth of microorganisms. Some examples of preservatives for use in the present formulations include: benzalkonium chloride (0.004-0.01%), chlorobutanol (up to 0.5%), phenylethyl alcohol (up to 0.5%), phenylmercuric acetate (0.002-0.004%), phenylmercuric nitrate (0.002-0.004%), and thimerosal (up to 0.01%). The percentages are expressed as weight percentage of the formulation.

Among these preservatives benzalkonium is preferred. It not only inhibits the growth of microorganisms but also enhances drug absorption. Therefore, it serves dually as a preservative and an absorption enhancer. Benzalkonium can be used only for cationic drugs.

Mercurial preservatives, including phenylmercuric acetate (PMA), phenylmercuric nitrate (PMN) and thimerosal, are useful for anionic drugs which are not compatible with benzalkonium chloride. Chlorobutanol and phenylethyl alcohol are antimicrobial as well as local anesthetics.

In one aspect, the formulations of this invention comprise benzalkonium chloride as the preservative. The benzalkonium chloride may be present from about 0.005% to about 0.02% by weight of the formulation. In some aspects, the benzalkonium chloride is present at about 0.01% by weight of the formulation.

Viscosity enhancers are used to increase the viscosity of ophthalmic solutions to prolong the drug actions and to increase the bioavailability of ocular formulations. Further, polymeric viscosity enhancers help reduce the friction between the cornea and the eyelids, and reduce corneal dryness. Polymers also stabilize ocular suspensions to prevent drug particles from precipitating out. They assure uniformity, stability and high quality suspension eye drops. The normal viscosity of ophthalmic solutions is in the range of 12-15 centipoise (cps).

Some exemplary ophthalmic viscosity enhancers that can be used in the present formulation include: carboxymethyl cellulose sodium (0.2-2.5%); methylcellulose (0.2-2.5%); hydroxypropyl cellulose (0.2-2.5%); hydroxypropylmethyl cellulose (0.2-2.5%); hydroxyethyl cellulose (0.2-2.5%); polyethylene glycol 300 (0.2-1.0%); polyethylene glycol 400 ( 0.2-1.0%); polyvinyl alcohol (0.1-4.0%); and providone (0.1-2.0%). The percentages are expressed as weight percentage of the formulation.

Some natural products, such as veegum, alginates, xanthan gum, gelatin, acacia and tragacanth, may also be used to increase the viscosity of ophthalmic solutions.

In one aspect, the formulations of the present invention comprise polyvinylpyrrolidone from about 0.1% to about 3% by weight as the viscosity enhancer. In some aspects, the polyvinylprrolidone is present from about 1% to about 2% by weight of the formulation. In some other aspects, the polyvinylpyrrolidone is present at about 1.5% by weight of the formulation.

While the present ocular formulations have enhanced ocular absorption due to its employing a carboxylic acid as described above, the absorption may be further increased by using surfactants. The surfactants may also help increase drug solubility in the formulation. Nonionic surfactants may be preferred in the present formulations because of their low incidence of eye toxicity. One particularly preferred surfactant in the present formulations is benzalkonium chloride. It is an effective absorption enhancer as well as an antimicrobial agent. Other surfactants that may be used include: polysorbate 20, polysorbate 40 stearate, alkyl aryl polyethyl alcohol, polyoxypropylene-polyoxyethylenediol, dinoctyl sodium sulfosuccinate etc.

The final formulation should be sterile, essentially free of foreign particles, and have a pH that allows for optimum drug stability.

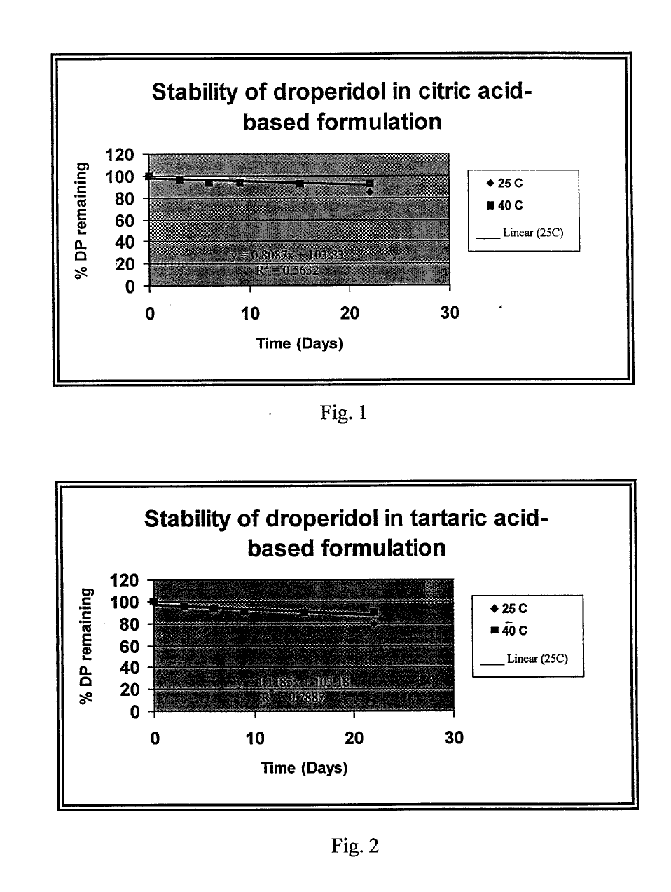

The formulations of the present invention are stable. When evaluated using accelerated stability tests at 40° C., the data indicated that the citric acid formulations are stable for about 74 days and the tartaric acid formulations are stable for about 18 days, at 25° C. See FIGS. 1-4. Since many ocular formulations are traditionally refrigerated until use, the shelf-life of these formulations in practice can be extended significantly.

E. Methods of Making

Typically, the formulations of the subject invention are prepared as solutions, suspensions, ointments, creams, gels, or ocular delivery devices such as drug-impregnated solid carriers that are inserted into the eye. A variety of polymers can be used to formulate ophthalmic drug carriers. Saettone, M. F., et al., J. Pharm. Pharmocol (1984) 36:229, and Park, K. et al., in Recent Advances in Drug Delivery Systems, Anderson et al., eds., Plenum Press (1984) 163-183, describe such polymers, the disclosures of which are incorporated herein by reference in their entirety. Drug release is generally effected via dissolution or bioerosion of the polymer, osmosis, or combinations thereof. The device should be formulated to release the drug at a rate that does not significantly disrupt the tonicity of tear fluid.

Several matrix-type delivery systems can be used with the subject invention. These systems are described in detail in Ueno et al., “Ocular Pharmacology of Drug Release Devices”, in Controlled Drug Delivery, Bruck, ed., vol. II, Chap 4 CRC Press Inc. (1983), the disclosure of which is incorporated herein by reference. Such systems include hydrophilic soft contact lenses impregnated or soaked with the desired drug, as well as biodegradable or soluble devices that need not be removed after placement in the eye. These soluble ocular inserts can be composed of any degradable substance that can be tolerated by the eye and that is compatible with the drug to be administered. Such substances include but are not limited to poly(vinyl alcohol), polymers and copolymers of polyacrylamide, ethylacrylate, and vinylpyrrolidone, as well as cross-linked polypeptides or polysaccharides, such as chitin.

Ophthalmic ointments will include a base, generally composed of white petrolatum and mineral oil, often with anhydrous lanolin. Polyethylene-mineral oil gel is also satisfactory, as are other substances that are non-irritating to the eye, permit diffusion of the drug into the ocular fluid, and retain activity of the medicament for a reasonable period of time under storage conditions. If suspensions are used, the particle sizes therein should be less than 10 μm to minimize eye irritation. Furthermore, if solutions or suspensions are used, the amount delivered to the patient should not exceed 50 μl, preferably 25 μl or less, to avoid excessive spillage from the eye.

Liquid dosage forms can, for example, be prepared by dissolving, dispersing, etc. a drug and an adjuvant in a vehicle, such as, for example, water, saline, aqueous dextrose, glycerol, ethanol, and the like, to thereby form a solution or a dispersion. If desired, the pharmaceutical formulation to be administered may also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, etc. Actual methods of preparing such dosage forms are known, or will be apparent, to those skilled in this art, for example, see Remington, supra, Chapter 89.

The formulations of the present invention may also be formulated in gel. At least one of the acids, and an ocular drug may be dissolved in a mixture of ethanol, water and propylene glycol in a volume ratio of, for example, 40:40:20, respectively. A gelling agent such as hydroxyethylcellulose, hydroxypropylcellulose, hydroxypropylmethylcellulose or ammoniated glycyrrhizinate may then be added to the mixture with agitation. The preferred concentration of the gelling agent may range from 0.1 to 4 percent by weight of the total formulation. Methods of preparing gels are known, or will be apparent, to those skilled in this art. See, for example, Remington, supra, Chapter 86.

One of the ordinary skill in the art readily appreciates that the ocular formulations of this invention are prepared under aseptic (sterile) conditions as required for ocular administration.

The amount of drug in the formulation will depend on the subject being treated, the manner of administration and the judgment of the prescribing physician.

F. Methods of Using

A wide variety of systemic and ocular conditions such as inflammation, infection, cancerous growth, may be prevented or treated using the drug delivery devices of the present invention. More specifically, ocular conditions such as ischemic retinal degeneration, glaucoma, proliferative vitreoretinopathy, diabetic retinopathy, uveitis, keratitis, cytomegalovirus retinitis, herpes simplex viral and adenoviral infections can be treated or prevented.

Ischemic retinal degeneration, or degeneration of the central part of the retina, is the second leading cause of blindness among people of all ages. This ischemic retinal degeneration is caused by various diseases, including diabetic retinopathy, glaucoma, sickle cell retinopathy, vascular abnormalities, obstructive arterial and venous retinopathies, venous capillary insufficiency, hypertensive retinopathy, inflammation, tumors, retinal detachment, etc.

The retina is supplied with oxygen and nutrients by two vascular systems, one within the retina itself (central retinal artery) and one in the choroid (posterior ciliary artery). Interruption or impairment of either system leads to degeneration of the retina and ultimately to loss of vision. There are many diseases and conditions that affect retinal circulation and nutritional supply. Early improvement in blood flow or nutrient supply to the retina in some of these diseases and throughout the time course of others might be the key to slowing vision loss or eliminating it altogether.

Various dopamine antagonists such as haloperidol, trifluperidol, moperone, and domperidone have been shown to lower intraocular pressure. See, for example, U.S. Pat. Nos. 4,565,821 and 4,772,616, which are incorporated herein by reference. The use of the dopamine antagonists haloperidol, moperone, trifluperidol, clofluperol, pipamperone and lemperone in the treatment of ocular hypertension and glaucoma is also described. Chiou, Ophthal. Res. 16:129-134 (1984). The methods described in the above patents and publications can be used to evaluate the therapeutic effectiveness of the present formulations.

Briefly, the method for studying the effects of droperidol on ocular blood flow comprises the following. Rabbits may be anesthetized and half the initial dose can be topically applied at one hour intervals afterward to maintain adequate anesthesia. The left ventricle of the heart is cannulated through the right carotid artery for microsphere injection, and the femoral artery was cannulated for blood sampling. The blood flow is measured with colored microspheres at −30 min for normal ocular blood flow and at 0 min for ocular blood flow with an intraocular pressure of 40 mm Hg. Droperidol eyedrops is instilled topically at time 0 min, and the blood flow is determined at 30, 60, 120, and 180 minutes thereafter. At each injection of microspheres, blood samples are taken from the femoral artery for exactly 60 seconds immediately after the injection of the microspheres as a reference.

After the last injection of the microspheres and the collection of blood samples, the animals are euthanized. The eyes are enucleated and dissected into the retina, choroid, iris and ciliary body. The tissue samples are weighed The blood sample is collected in a heparinized tube, and the volume is recorded. The number of microspheres are measured by using the appropriate instrumentation as described.

The blood flow of each tissue (iris, ciliary body, retina and choroid) at a certain time point can be calculated from the following equation:

Qₘ=(Cₘ.x.Qᵣ)/Cr

where Qₘ is the blood flow of a tissue in terms of μl/min/mg. Cₘ is the microsphere count per mg of tissue, Qᵣ is the flow rate of blood sample in terms of μl/min, and Cr is the microsphere count in the referenced blood sample.

Accordingly, the present invention provides a method to increase blood flow to the retina or choroid, to reduce intraocular pressure, or to treat or prevent visual deterioration associated with decreased retinal or choroidal blood flow or increased intraocular pressure, which method comprises ocularly administering a formulation comprising a therapeutically effective amount of a dopamine antagonist, a carboxylic acid to maintain the pH of the formulation from about 4.5 to about 7.5 to a subject having decreased retinal or choroidal blood flow or increased intraocular pressure. The various aspects of the formulations are described above.

The decreased retinal or choroidal blood flow may be due to low pressure glaucoma, ischemic retinal degeneration, or age-related macular degeneration. The ischemic retinal degeneration may be caused by a disease such as diabetic retinopathy, glaucoma, sickle cell retinopathy, vascular abnormalities, obstructive arterial and venous retinopathies, venous capillary insufficiency, hypertensive retinopathy, inflammation, tumors, or retinal detachment.

Thus, in one specific aspect, the above methods comprise using a formulation comprising a dopamine antagonist such as metoclopromide, loxapine, or droperidol, and an acid such as tartaric acid, lactic acid or citric acid and the pH of the formulation is about 5.5.

G. Administration

The above methods can be practiced by administering the formulations of this invention in a dosage form such as a solution, dispersion, cream, ointment, gel, or film. The above formulation may also be administered through the use of an ocular delivery device, such as an ocusert. Several such devices have been described in the art. See, for example, the U.S. Pat. No. 5,660,851.

For topical administration, i.e. application of solutions, suspensions, ointments, gels, etc. directly to the eye, the formulation may contain 0.01-20.0% active ingredient, preferably 0.1-5.0%. An effective amount for the purposes of preventing or treating visual deterioration may range from about 0.01 to 0.1 mg/kg. The compound may be administered on a convenient schedule as dictated by the required therapeutic levels, duration of treatment, patient compliance, etc. Thus, the dosage can be every 4-8 hours, or every 12 hours, or every 24 hours, or once a week.

The subject compounds can also be administered by implantation of a slow-release or sustained-release system, such that a constant level of dosage is maintained. For a review of these sustained release systems see Ueno, et al., “Ocular Pharmacology of Drug Release Devices”, in Controlled Drug Delivery, Bruck, ed., vol. II, Chap 4, CRC Press Inc. (1983).

An effective amount for the purposes of preventing or treating visual deterioration is usually in the range of 0.01-0.5 mg/kg.

The following are illustrative examples of formulations according to this invention. Although the examples utilize only selected compounds and formulations, it should be understood that the following examples are illustrative and not limiting. Other aspects, advantages and modifications within the scope of the invention will be apparent to those skilled in the art to which the invention pertains.

### EXAMPLES

A. Materials

Droperidol was purchased commercially from Janssen Pharmaceuticals Inc. (Piscataway, N.J.). PVP, benzalkonium chloride, and all other ingredients are purchased from commercial sources.

### Example 1

### Droperidol Formulation

The formulations in this study were prepared using the formula below:

| | |
|---|---|
| Droperidol | 0.5% |
| PVP | 1.5% |
| Benzalkonium chloride | 0.01% |
| Sodium diedetate | 0.01% |
| Sodium chloride | 0.5% |
| 0.1 N acid | q.s. to pH 5.5 |
| Water | q.s |

All ingredients were mixed together prior to titration with the 0.1N acid to pH of 5.5. The acids used were lactic acid, tartaric acid and citric acid.

### Example 2

### Droperidol Formulation Stability

Droperidol formulations in citric acid and tartaric acid were prepared according to Example 1. The formulations contained 0.01% each of benzalkonium chloride and EDTA and were sterile filtered. The stability testings were performed at 25° C. and at 40° C. according to methods well-known in the art. For example, see Remington, supra, Chapter 18. The data are presented graphically in FIGS. 1-4.

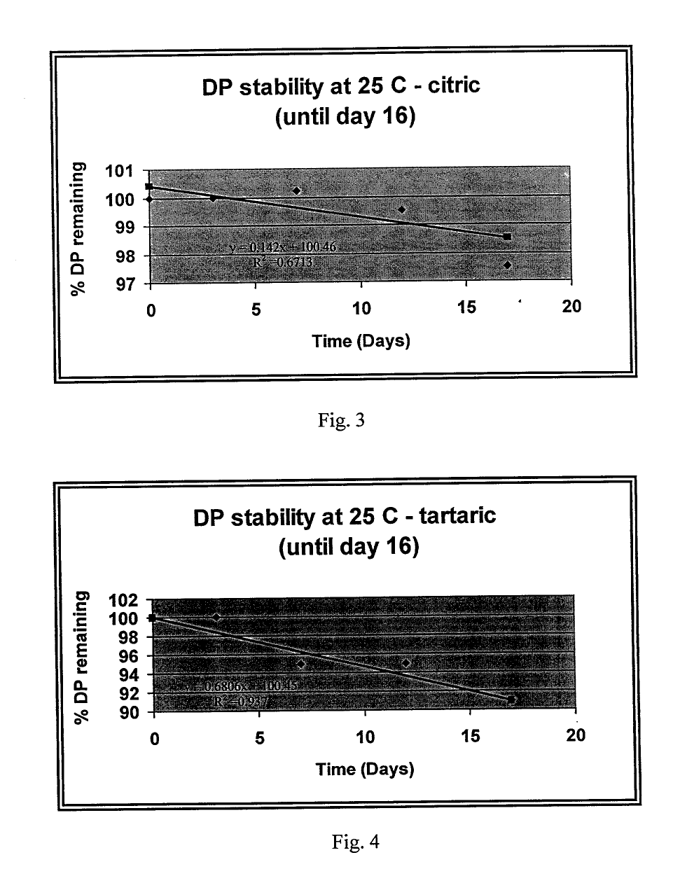

FIGS. 1 and 2 show the stability data for tartaric acid and citric acid formulations at both 25° C. and at 40° C. The shelf life (t₉₀) of droperidol in citric acid and tartaric acid-based formulations are ˜17 days and ˜12 days, respectively at 40° C. It should be noted that the shelf lives may be attributable to physical instability rather than chemical degradation. FIGS. 3 and 4 show the data for 16 days. The data show that the shelf life (t₉₀) of droperidol in citric acid- and tartaric acid-based formulations are ˜74 days and ˜18 days, respectively, at 25° C. These values have been calculated from the linear regression of data points obtained for storage of the drug at 40° C.

### Example 3

### Topical Droperidol Bioavailability in Rabbit Eye

The bioavailability of droperidol formulations as described above were measured using established techniques in the art. Droperidol ophthalmic solutions comprising citric acid, or tartaric acid were topically applied to eyes of NewZealand albino rabbits. Phosphate Buffered Saline (of pH 7.4) was used as a control. The concentration of droperidol was measured in both cornea and aqueous humor for each formulation at 30 minutes and 60 minutes after the administration. The results are shown in Table 1 below.

TABLE 1
Ocular Bioavailability Data

| | | | CONCENTRATIONS | | | |
|---|---|---|---|---|---|---|
| | | | Aqueous humor | | Cornea | |
| Formulation | Time | Rabbit | (ng DP per | | (ng DP per | |
| Base | (min) | Eye ID | uL fluid) | average | gram tissue) | average |
| Citric acid | 30 | 1R | 0.58 | | 1953 | |
| Citric acid | 30 | 1L | * | | 1948 | |
| Citric acid | 30 | 2R | 0.57 | | 1831 | |
| Citric acid | 30 | 2L | 0.55 | | 2091 | |
| Citric acid | 30 | 3R | 0.53 | | 1656 | |
| Citric acid | 30 | 3L | 0.54 | 0.55 | 1613 | 1849 |
| Citric acid | 60 | 4R | 0.55 | | 2056 | |
| Citric acid | 60 | 4L | 0.54 | | 2502 | |
| Citric acid | 60 | 5R | 0.54 | | 3013 | |
| Citric acid | 60 | 5L | 0.55 | | 2370 | |
| Citric acid | 60 | 6R | 0.56 | | 2266 | |
| Citric acid | 60 | 6L | 0.58 | 0.55 | 2231 | 24.06 |
| Tartaric acid | 30 | 7R | 0.54 | | 2257 | |
| Tartaric acid | 30 | 7L | 0.54 | | 2055 | |
| Tartaric acid | 30 | 8R | 0.54 | | 2153 | |
| Tartaric acid | 30 | 8L | 0.54 | | 2264 | |
| Tartaric acid | 30 | 9R | 0.55 | | 2420 | |
| Tartaric acid | 30 | 9L | 0.54 | 0.54 | 2545 | 2282 |
| Tartaric acid | 60 | 19R | 0.55 | | 2076 | |
| Tartaric acid | 60 | 10L | 0.54 | | 1867 | |
| Tartaric acid | 60 | 11R | 0.63 | | 2628 | |
| Tartaric acid | 60 | 11L | 0.56 | | 1660 | |
| Tartaric acid | 60 | 12R | 0.60 | | 1823 | |
| Tartaric acid | 60 | 12L | 0.55 | 0.57 | 2041 | 2019 |
| | % DP | | | | | |
| Citric acid | 0.0307 | | | | | |
| Tartaric acid | 0.0329 | | | | | |

*sample handling problem

The data were further summarized, along with additional characteristics for each formulation as below in Table 2.

TABLE 2
Summary Data of Formulations

| | Formulation I | Formulation II | Formulation III |
|---|---|---|---|
| | PBS | tartaric | citric |
| Acid | 0.0191 | 0.0061 | 0.065 |
| % Droperidol | solution | solution | solution |
| pH | 5.5 | 5.5 | 5.5 |
| rabbits/time pt. | 3 | 3 | 3 |
| easily | yes | yes | yes |
| sterilizable? | | | |
| Irritates rabbit? | no | no | no |
| approved | yes | yes | yes |
| excipients? | | | |
| 30 min, ng/μl | .43 | 0.29 | 0.29 |
| 60 min, ng/μl | 0.29 | 0.41 | 1.25 |
| Rank at 60 min | 4 | 3 | 1 |

These results indicate that either citric acid or tartaric acid formulation base can be used with equal efficacy in droperidol absorption into the eyeball, i.e., aqueous humor, not cornea.

Formulation I showed faster absorption with peak bioavailability at 30 minutes whereas Formulation II was slower with peak availability at 60 minutes. The best one is Formulation III which allowed the highest bioavailability at 60 minutes after instillation of eyedrops. Since citric acid formulation allows droperidol concentration to be increased to 0.5% (from 0.0061%) the bioavailability and drug efficacy of droperidol can be enhanced markedly.

### Example 4

### Eye Irritation

50 μl of 1% droperidol was used to test for eye irritation in New Zealand white rabbit eyes using the standard procedure of the Draize Test. J. Draize et al., J. Pharmacol. Exp. Ther., 82:377-390 (1944). Eyedrops were instilled in the eyes, and the responses of the eyes were examined with a slit lamp biomicroscope. Eye irritation was either absent or insignificant in each case.

Modifications of the above described modes for carrying out the invention that are obvious to persons of skill in the art to which the invention pertains are intended to be within the scope of the invention.

What is claimed:

1. A formulation for ocular delivery comprising an ocular drug and a carboxylic acid in an amount sufficient to maintain the pH of the formulation from about 4.5 to about 7.5.

2. The formulation of claim 1, wherein the ocular drug is a dopamine antagonist.

3. The formulation of claim 1, wherein the pH of the formulation is from about 5.0 to about 6.0.

4. The formulation of claim 1, wherein the pH of the formulation is from about 5.2 to about 5.7.

5. The formulation of claim 1, wherein the pH of the formulation is about 5.5.

6. The formulation of claim 2, wherein the dopamine antagonist is a butyrophenone or a phenothiazine or a mixture thereof.

7. The formulation of claim 6, wherein the dopamine antagonist is droperidol.

8. The formulation of claim 2, wherein the dopamine antagonist is metoclopromide.

9. The formulation of claim 2, wherein the dopamine antagonist is loxapine.

10. The formulation of claim 1, further comprising at least one adjuvant.

11. The formulation of claim 10, wherein the adjuvant is a viscosity enhancer, a preservative, tonicity adjuster, an absorption enhancer, a stabilizer, or a mixture thereof.

12. The formulation of claim 11, wherein the viscosity enhancer comprises polyvinylpyrrolidone.

13. The formulation of claim 1, wherein the carboxylic acid is a hydroxymonocarboxylic acid having the following chemical formula:
R₁(CR₂OH)ₘ(CH₂)ₙCOOH, wherein
R₁ and R₂ are selected from the group consisting of hydrogen, alkyl, aralkyl and aryl, wherein
the alkyl, aralkyl and aryl groups may be saturated or unsaturated, and straight or branched and the alkyl group has from 1 to 25 carbon atoms, the aralkyl group has from 7 to 25 carbon atoms, and the aryl group has from 6 to 25 carbon atoms;
m is an integer of from 1 to 9, and n is an integer of from 0 to 23, or a D, L and DL isomer, or a mixture thereof.

14. The formulation of claim 13, wherein the hydroxymonocarboxylic acid is selected from the group consisting of glycolic acid, lactic acid, methyllactic acid, 2-hydroxybutanoic acid, mandelic acid, atrolactic acid, phenyllactic acid, glyceric acid, 2,3,4-trihydroxybutanoic acid, 2,3,4,5-tetrahydroxypentanoic acid, 2,3,4,5,6-pentahydroxyhexanoic acid, 2-hydroxydodecanoic acid, 2,3,4,5,6,7-hexahydroxyheptanoic acid, benzillic acid, 4-hydroxymandelic acid, 4-chloromandelic acid, 3-hydroxybutanoic acid, 4-hydroxybutanoic acid, 2-hydroxyhexanoic acid, 5-hydroxydodecanoic acid, 12-hydroxydodecanoic acid, 10-hydroxydecanoic acid 16-hydroxyhexadecanoic acid, 2-hydroxy-3-methylbutanoic acid, 2-hydroxy-4-methylpentanoic acid, 3-hydroxy-4-methoxymandelic acid, 4-hydroxy-3-methoxymandelic acid, 2-hydroxy-2-methylbutanoic acid, 3-(2-hydroxyphenyl) lactic acid, 3-(4-hydroxyphenyl) lactic acid, hexahydromandelic acid, 3-hydroxy-3-methylpentanoic acid, 4-hydroxydecanoic acid, 5-hdroxydecanoic acid and aleuritic acid.

15. The formulation of claim 1, wherein the carboxylic acid is a hydroxydicarboxylic acid having the following formula: 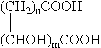
wherein m is an integer of from 1 to 9, and n is an integer of from 0 to 23, or a D, L and DL isomer or a mixture thereof.

16 The formulation of claim 15, wherein the hydroxydicarboxylic acid is selected from the group consisting of tartronic acid, malic acid, tartaric acid, arabiraric acid, ribaric acid, xylaric acid, lyxaric acid, saccharic acid, mucic acid, mannaric acid, gularic acid, allaric acid, altraric acid, idaric acid and talaric acid.

17. The formulation of claim 1, wherein the carboxylic acid is a hydroxyacid having the following formula:
R(OH)ₘ(COOH)ₙ
wherein
R is selected from the group consisting of hydrogen, alkyl, aralkyl and aryl, wherein
the alkyl, aralkyl and aryl groups may be saturated or unsaturated, and straight or branched, and the alkyl group has from 1 to 25 carbon atoms, the aralkyl group has from 7 to 25 carbon atoms, and the aryl group has from 6 to 25 carbon atoms;
m is an integer of from 1 to 9, and
n is an integer of from 1 to 9, or a D, L and DL isomer or a mixture thereof.

18. The formulation of claim 17, wherein the hydroxyacid is selected from the group consisting of citric acid, isocitric acid, citramalic acid, agaricic acid, quicnic acid, glucuronic acid, galacturonic acid, hydroxypyruvic acid, ascorbic acid, dihydroascorbic acid, dihydroxytartaric acid, 2-hydroxy-2-methylbutanoic acid, 1-hydroxy-1-cyclopropane carboxylic acid, 3-hydroxy-2-aminopentanoic acid, tropic acid, 4-hydroxy-2,2-diphenylbutanoic acid, 3-hydroxy-3-methylglutaric acid and 4-hydroxy-3-pentenoic acid.

19. A formulation for ocular delivery comprising a dopamine antagonist, a carboxylic acid in an amount sufficient to maintain the pH of the formulation from about 4.5 to about 7.5, wherein the dopamine antagonist is metoclopromide, loxapine, or droperidol, and the acid is tartaric acid, lactic acid or citric acid and the pH of the formulation is about 5.5.

20. The formulation of claim 1, which has a shelf-life of at least 14 days at 25° C.

21. A method to increase blood flow to the retina or choroid, to reduce intraocular pressure, or to treat or prevent visual deterioration associated with decreased retinal or choroidal blood flow or increased intraocular pressure, which method comprises ocularly administering a formulation comprising a therapeutically effective amount of a dopamine antagonist and a carboxylic acid in an amount sufficient to maintain the pH of the formulation from about 4.5 to about 7.5 to a subject having decreased retinal or choroidal blood flow or increased intraocular pressure.

22. The method of claim 21, wherein the decreased retinal or choroidal blood flow is due to low pressure glaucoma, ischemic retinal degeneration, or age-related macular degeneration.

23. The method of claim 22, wherein the ischemic retinal degeneration is caused by a disease selected from the group consisting of diabetic retinopathy, glaucoma, sickle cell retinopathy, vascular abnormalities, obstructive arterial and venous retinopathies, venous capillary insufficiency, hypertensive retinopathy, inflammation, tumors, and retinal detachment.

24. The method of claim 21, wherein the formulation is in a solution, dispersion, cream, ointment, gel, or film.

25. The method of claim 21, wherein ocular administration is accomplished through the use of an ocular delivery device.

26. The method of claim 21, wherein the dopamine antagonist is metoclopromide, loxapine, or droperidol, and the acid is tartaric acid, lactic acid or citric acid and the pH of the formulation is about 5.5.
